(19) United States
(12) Patent Application Publication
(10) Pub. No.: US 2022/0226414 A1
(43) Pub. Date: Jul. 21, 2022

(54) COVID-19 Treatment

(71) Applicants: Firman Zulkifli Amin, Jakarta Timur (ID); Mila Kurnia Sari, Jakarta Timur (ID)

(72) Inventors: Firman Zulkifli Amin, Jakarta Timur (ID); Mila Kurnia Sari, Jakarta Timur (ID)

(21) Appl. No.: 17/485,435

(22) Filed: Sep. 26, 2021

(30) Foreign Application Priority Data
Jan. 16, 2021 (ID) S00202100379

(51) Int. Cl. A61K 36/71; A61P 31/14

(52) U.S. Cl. CPC A61K 36/71; A61P 31/14

(57) ABSTRACT

The disease due to infection with the severe acute respiratory syndrome coronavirus 2 (SARS-CoV-2) referred to as Coronavirus disease 2019 (COVID-19) can be treated by using a product in the form of Semen Nigella Sativa Oil (oil from Habbatus sauda seed or black cumin seed) as an ingredient or drug for COVID-19 treatment that is taken orally. The consumption is carried out daily, three times a day, with each consumption the composition of the oil content of the black cumin seeds is composed of about 450 mg, according to weight, until recovered from COVID-19.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND OF THE INVENTION

This invention relates to the treatment of Coronavirus disease 2019 (COVID-19) using a product in the form of Semen Nigella Sativa Oil (oil from Habbatus sauda seed or black cumin seed) which is consumed orally in effective levels as an ingredient or drug for the treatment of COVID-19 with in each consumption the composition of the oil content of the black cumin seeds is composed of about 450 mg, by weight, accompanied by an effective level of preservative namely Sodium Ethylparaben composed of about 0.87 mg and Sodium Propylparaben composed of about 0.435 mg, by weight, and repeat the consumption every day, three times every day, and continue the treatment until recovered from COVID-19 where consuming it increases the CT Value on the results of the reverse transcription-polymerase chain reaction (RT-PCR) swab test from the previous CT value<40 which indicates positive confirmation of COVID-19 to CT value>=40 which shows negative confirmation of COVID-19 within about two to three weeks.

Nigella sativa, which is also referred to as Habbatus sauda or black cumin, is a “miracle ingredient” from the Ranunculaceae family which is well known as a medicine with a broad spectrum of pharmacological benefits. Black cumin has the potential to integrally complement the core basic needs of the COVID-19 treatment that is still uncertain specifically in terms of what ingredients or drugs and in what composition and at what levels are actually effective for the treatment of COVID-19 (Amin, F. Z. et. al., “Nigella sativa (Habbatus sauda): The Perspectives For COVID-19 Treatment,” The Internet Journal of Infectious Diseases, 18(1), May 2020). The integral role that black cumin plays in the core basic needs of COVID-19 treatment, we found, can be understood based on the two phases of the immune response caused by COVID-19 infection. Understanding the immune response that occurs in the mechanism of COVID-19 caused by infection with severe acute respiratory syndrome coronavirus 2 (SARS-CoV-2) has brought our attention to a more specific need in the potential of targeted drugs for the treatment of COVID-19 patients.

The immunomodulatory ability of black cumin, as a single-alternative therapy has been shown to increase viral load in patients with Hepatitis C virus (HCV) who are not eligible for therapy with interferon (IFN)/ribavirin, explained by a significant increase in macrophages and CD4+ T cell along with significant decrease in viral titers and increased levels of IFN-gamma (IFN-γ) in the blood, we found, this indicates the potential of black cumin to play a role in the phase 1 immune response which includes asymptomatic and incubation periods in the first stage of the disease course of COVID-19 and a period of symptomatic disease is not severe in the second stage, which emphasizes the basic need for immune protection by treatment based on immune enhancement, either with IFN-activating or vaccines.

The mechanism of in vitro antiviral activity of black cumin has been further investigated suggesting a general decrease in coronavirus replication and viral load. What stands out, black cumin is known to be safe and its side effects are mediocre. Therefore, black cumin is considered to be used for self-medication, as a consideration of its added value in the perspective of preventive therapy in connection with the search for useful immunizations in the field of SARS-CoV-2 vaccine research. In addition, the potential ability of black cumin to exert the required anti-inflammatory effect by activating IFN-γ, we found, is also useful for another core basic need of COVID-19 treatment, namely in patients with severe symptoms due to lack of this activation ability because the T cell is not well activated by SARS-CoV-2 infection in phase 2 of the immune response, which is dominated by cytokine release syndrome (CRS) causing cytokine storms and, consequently, lung damage due to lung inflammation, which describes the severe condition in the third stage of the disease course.

### BRIEF SUMMARY OF THE INVENTION

We have found that the disease caused by SARS-CoV-2 infection known as COVID-19 can be treated by using a product in the form of Semen Nigella Sativa Oil (oil from Habbatus sauda seed or black cumin seed) which is taken orally. The oil from black cumin seeds used in this specification refers to the levels that are effective as ingredients and drugs for the treatment of COVID-19 with each consumption of the composition of the oil content of the black cumin seeds composed of about 450 mg, by weight, accompanied by an effective preservative level, namely Sodium Ethylparaben is composed of about 0.87 mg and Sodium Propylparaben is composed of about 0.435 mg, by weight, and repeat taking it every day, three times every day, and continue the treatment until recovering from COVID-19 where taking it increases the CT Value (CT Value) on the results of the reverse transcription-polymerase chain reaction (RT-PCR) swab test, from the previous CT value<40 which indicates positive confirmation of COVID-19 to CT value>=40 which indicates negative confirmation of COVID-19 within a period of approximately two to three weeks.

### BRIEF DESCRIPTION OF THE SEVERAL VIEWS OF THE DRAWING

FIG. 1 shows the treatment of COVID-19 using Semen Nigella Sativa Oil (oil from Habbatus sauda seed or black cumin seed) in effective composition and levels as an ingredient or drug that is taken orally by causing activation or increasing levels of interferon-gamma (IFN-γ) in the blood that plays a role both when asymptomatic and until severe symptoms by increasing the CT Value on the results of the reverse transcription-polymerase chain reaction (RT-PCR) swab test from the previous CT value<40 which indicates positive confirmation for COVID-19 becomes CT Value>=40 which indicates negative confirmation of COVID-19 within a period of about two to three weeks.

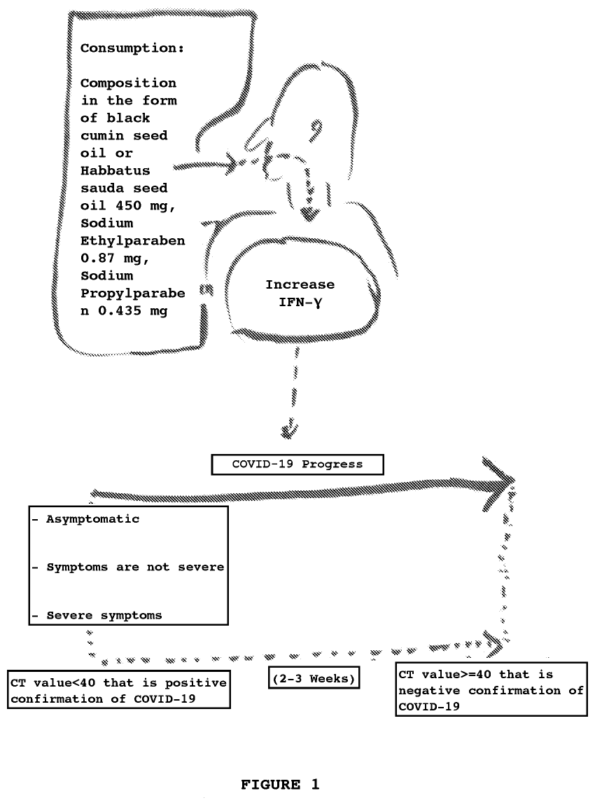

### DETAILED DESCRIPTION OF THE INVENTION

Treatment of Coronavirus disease 2019 (COVID-19) is to use a product in the form of Semen Nigella Sativa Oil (oil from Habbatus sauda seed or black cumin seed) which is consumed orally in effective levels as an ingredient or drug for the treatment of COVID-19.

In each consumption, the composition of the oil content of black cumin seeds is composed of about 450 mg, by weight, accompanied by an effective preservative level, namely Sodium Ethylparaben composed of about 0.87 mg and Sodium Propylparaben composed of about 0.435 mg, by weight, and repeat consumption. daily, three times a day, and continue the treatment until recovered from COVID-19.

This consumption increases the CT Value (CT value) on the results of the reverse transcription-polymerase chain reaction (RT-PCR) swab test examination from the previous CT value<40 which indicates positive confirmation of COVID-19 to CT value>=40 which indicates negative confirmation of COVID-19 within about two to three weeks.

The mechanism that occurs is causing activation or increasing levels of interferon-gamma (IFN-γ) in the blood which results in increased immunity that acts when asymptomatic and symptoms are not severe and produces the anti-inflammatory effect needed to play a role in times of severe symptoms that in these conditions there is an inability to activate interferon-gamma (IFN-γ) because the T cell is not activated properly.

1. Coronavirus disease 2019 (COVID-19) treatment is to use a product in the form of Semen Nigella Sativa Oil (oil from Habbatus sauda seed or black cumin seed) which is consumed orally in effective levels as an ingredient or drug for the treatment of COVID-19 with a composition in each consumption comprising the oil content of black cumin seeds that is composed of about 450 mg, by weight, accompanied by levels of an effective preservative, namely Sodium Ethylparaben is composed of about 0.87 mg and Sodium Propylparaben is composed of about 0.435 mg, by weight, and repeats consuming it every day, three times every day, and continue the treatment until recovered from COVID-19 where consuming it increases the CT Value of the reverse transcription-polymerase chain reaction (RT-PCR) swab test results from the previous CT value<40 which indicates a positive confirmation of COVID-19 becomes a CT value>=40 which indicates a negative confirmation of COVID-19 within a period of about two to three weeks based on the mechanism that occurs, namely causing activation or increasing levels of interferon-gamma (IFN-γ) in the blood which results in increased immunity that acts during asymptomatic and symptoms are not severe and produces the exertion of anti-inflammatory effects which is needed to play a role during severe symptoms in which there is an inability to activate interferon-gamma (IFN-γ) because T cell is not activated properly.
